(19) United States
(12) Patent Application Publication
(10) Pub. No.: US 2007/0017526 A1
(43) Pub. Date: Jan. 25, 2007

(54) Methods and apparatus for sub-glottic secretion collection

(75) Inventor: Amir Abolfathi, Woodside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: APMed Solutions, Inc., Woodside, CA (US)

(21) Appl. No.: 11/186,134

(22) Filed: Jul. 20, 2005

(51) Int. Cl. A61M 16/00

(52) U.S. Cl. 128/207.15

(57) ABSTRACT

A sub-glottic secretion collection device comprises a body, a baffle, and a port. The body is expandable to be deployed within a patient's trachea beneath the larynx. The baffle diverts secretions entering the trachea into a collection receptacle. The secretions may be aspirated through a port coupled to the collection receptacle. A relatively unobstructed air passageway through the device permits normal patient breathing while the secretions are being aspirated.

### BACKGROUND OF THE INVENTION

1. Field of the Invention

The present invention relates generally to medical methods and apparatus. More particularly, the present invention relates to methods and devices for collecting secretions in a patient airway to prevent or limit passage into the patient's lungs.

Aspiration pneumonia is a common but serious condition which can arise from a number of causative factors. Of particular interest to the present invention, aspiration pneumonia can result from obstruction of the lower airways caused by aspiration of fluids, secretions, and particulate matter in unconscious or semi-conscious patients. While acute obstructions can be treated by tracheal suction, long-term treatment and prevention is more difficult.

For these reasons, it would be desirable to provide methods and apparatus for continuously aspirating fluids and particulates which would otherwise collect in a patient's trachea and pass into the lower airways. It would be particularly desirable if such methods and apparatus allowed for substantially unimpaired breathing while permitting either or both continuous or periodic aspiration of collected materials. At least some of these objectives will be met by the inventions described hereinafter.

2. Description of the Background Art

U.S. Pat. No. 6,840,242 describes a tracheostomy aspiration suction tube for use with or without a tracheostomy cuff. Other tracheostomy tubes are described in U.S. Pat. Nos. 6,612,305; 6,575,944; 6,460,540; 5,957,978; 5,653,231; 5,392,775; 5,107,828; 5,056,515; 5,054,484; 4,979,505; 4,280,492; 4,278,081; and published U.S. application 2003/0037789. Certain endotracheal tubes are described in U.S. Pat. Nos. 6,843,250; 5,501,215; 5,311,864; 5,143,062; 5,067,497; 4,840,173; and 4,305,392.

### BRIEF SUMMARY OF THE INVENTION

The present invention provides devices, methods, and systems for tracheal aspiration which is useful, for example, for collecting and removing secretions to prevent or inhibit intrusion of the secretions into the lower airways and inhibit aspiration pneumonia and other conditions. The methods and apparatus provide a collection receptacle which is implantable in the sub-glottic region of the trachea beneath the larynx. The collection receptacle permits relatively unimpeded breathing by providing a bypass air passage while being connectable to an external aspiration source. In alternative embodiments, the present invention could provide for drainage of the collected secretions into the patient esophagus.

In a first specific aspect of the present invention, a sub-glottic secretion collection device comprises a body, a baffle disposed within the body, and a port opening from a receptacle in the body to allow collected secretions to drain or be aspirated from the receptacle. The body is deployable within the patient's trachea in the sub-glottic region beneath the larynx. The body is expandable from a low width (low profile) configuration to an expanded configuration which is anchorable within the target region of the trachea. The body may be self-expanding, e.g. being a scaffold or a stent-like structure which can be contained within an external constraint, such as a delivery tube, and allowed to self-expand within the trachea to its deployed configuration. Alternatively, the body could be balloon expandable, i.e. expandable by application of an internal, radially outward force, to permit deployment over a balloon or other expansion catheter. Typically, the self-expanding bodies will be formed from an elastic metal or polymer, such as a superelastic metal, such as Nitinol. In contrast, self-expanding bodies will be composed of malleable materials, such as found in balloon-expandable stents, or in some instances from coiled sheet structures employing ratchets or similar means for maintaining patency, such as described in U.S. Pat. Nos. 6,793,672; 5,824,052; and 5,441,515, the full disclosures of which are incorporated herein by reference. The sub-glottic secretion devices may further comprise a baffle or other means or structure disposed within the body to divert secretions toward the receptacle and away from a relatively unimpeded airflow pathway which permits the patient to breathe normally after the collection device has been deployed.

In the exemplary embodiment, the body comprises a generally cylindrical structure having an upper end and a lower end. The upper end is disposed immediately beneath the larynx when the device is deployed while the lower end is located more remotely from the larynx. A dividing wall within the interior of the cylindrical structure defines a collections receptacle on one side thereof. The bottom of the body is open on the other side of the wall, defining the flow path. The port which permits drainage and/or aspiration of collected secretions is attached near the bottom of the receptacle defined by the wall, and the baffle is placed over the wall to divert secretions into the receptacle by gravity.

The sub-glottic secretion collection devices may be deployed by first placing a guidewire through a percutaneous passage of the patient's neck into the trachea. The guidewire may then be guided upwardly past the larynx and out through the patient's mouth. The collection device, in a collapsed, low width configuration, may then be introduced over the guidewire so that the secretion drainage port is guided to the percutaneous neck passage. The device is then expanded or allowed to expand so that it anchors within the trachea to provide the desired collection receptacle and airflow pathway. The device may be deployed either by self-expansion (release from constraint) or by expanding a balloon or other expansion element therein.

In a second aspect of the present invention, methods for collecting secretions passing down a patient's trachea comprise implanting a diverter device in the trachea below the larynx. The secretions are then collected by the collection device in a receptacle positioned in the diverter device. Air inspiration and exhalation are allowed through a flow path past the receptacle in the diverter. Usually, the collected secretions will be actively aspirated by applying a vacuum through a collection port coupled to the collection receptacle. Alternatively, the secretions may be allowed to drain by gravity from the receptacle, either to an external location or in some instances, directly into the patient's esophagus. In the latter case, the drainage port will be oriented through a passage in the tissue structure which separates the trachea from the esophagus.

### BRIEF DESCRIPTION OF THE DRAWINGS

FIG. 1 illustrates the relationship between the trachea, larynx, and esophagus in the neck of a patient.

FIG. 2 illustrates an exemplary sub-glottic secretion collection device (FIG. 2) constructed in accordance with the principles of the present invention.

FIGS. 3A-3D illustrate deployment and connection of the collection device of FIG. 2 in the neck of a patient.

FIG. 4 illustrates an alternate deployment scheme for the sub-glottic secretion device where secretions are drained into the patient's esophagus.

### DETAILED DESCRIPTION OF THE INVENTION

The present invention is intended primarily to provide for continuous collection of secretions in the trachea of a patient to prevent or inhibit passage of the secretions into the lower airways. While the apparatus and methods will typically involve continuous aspiration of the collected secretions, in some instances the apparatus could be used to permit active ventilation of the patient as well. In other instances, the devices can provide for passive (gravity induced) drainage of secretions, either externally or into the patient's esophagus.

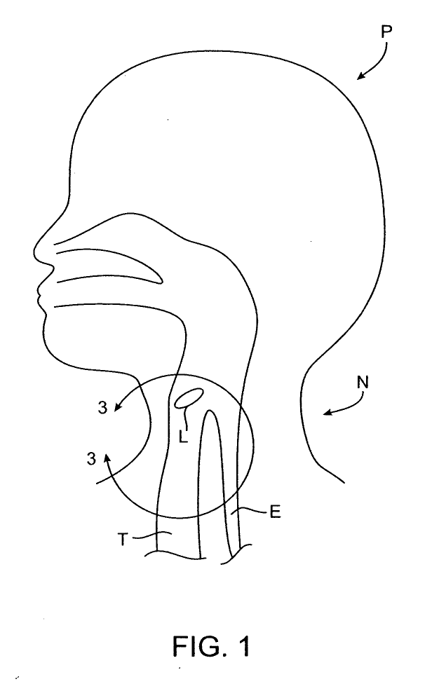

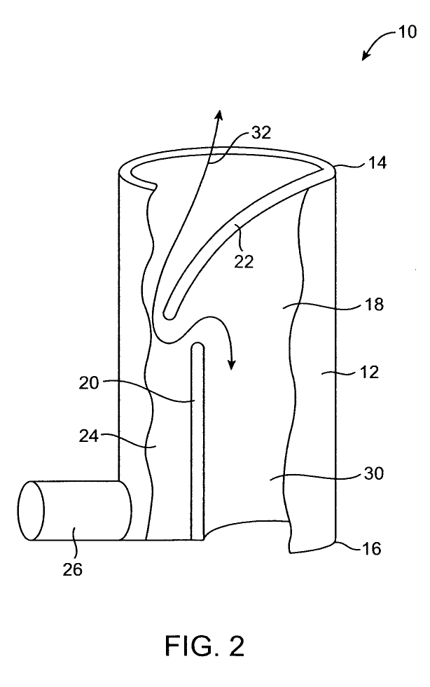

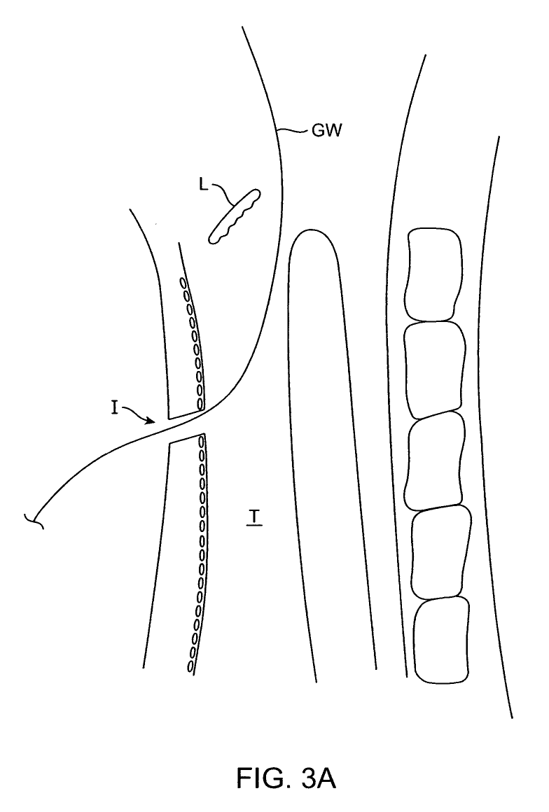

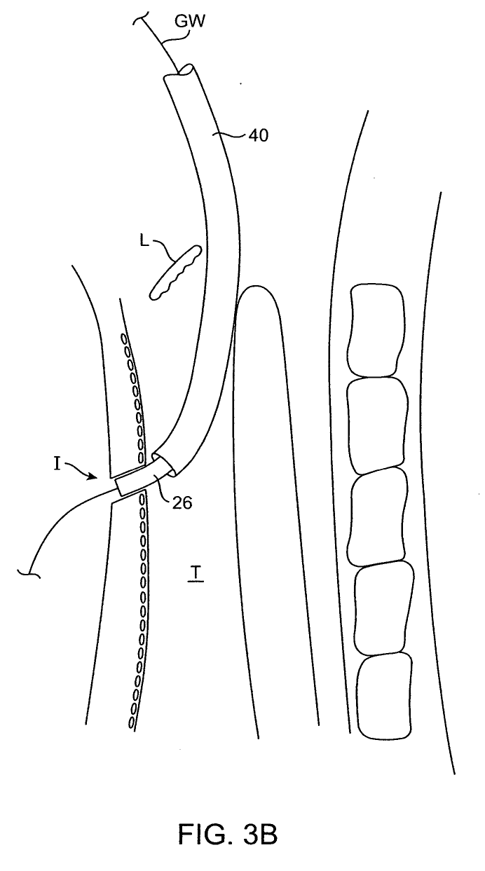

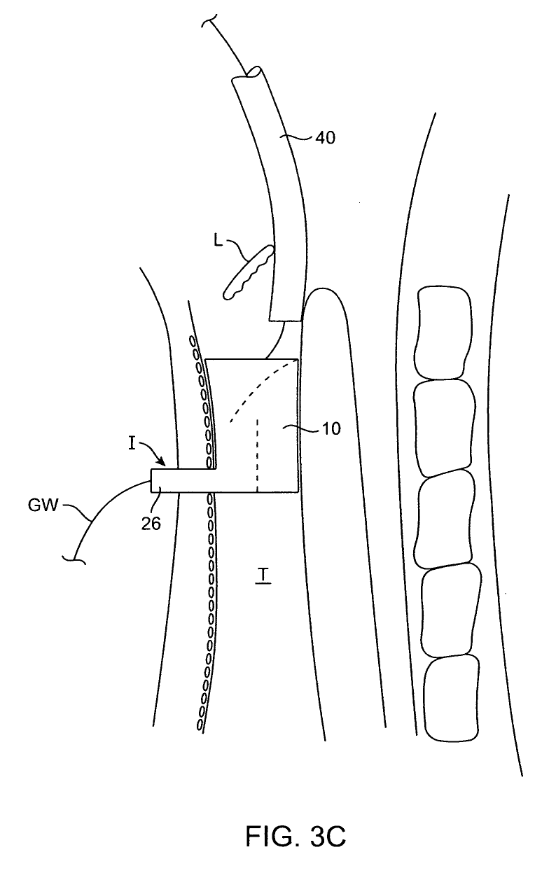

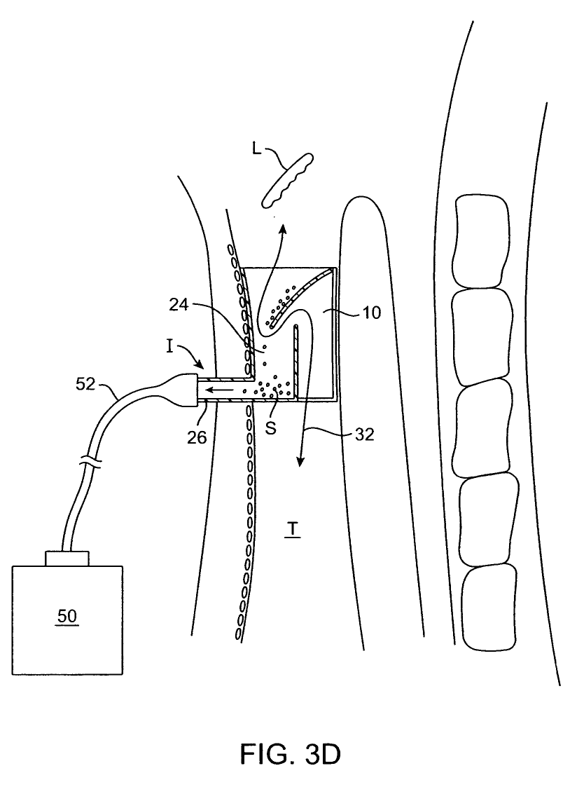

Referring now to FIG. 1, a neck region N of a patient P includes both the esophagus E for passing food and drink to the stomach and the trachea T for exchanging air with the lungs. The larynx L is located generally above the trachea, and incisions into the trachea to provide for positioning of an external drain port are generally made well below the larynx, as described in more detail below with reference to FIGS. 3A-3D.

An exemplary sub-glottic secretion collection device constructed in accordance with the principles of the present invention comprises a body 12 having an upper end 14 and lower end 16. The body 12 also provides an internal passage or lumen 18 having a dividing wall 20 in its lower half and a diverter structure 22 in its upper half. The dividing wall 20 defines a collection receptacle 24 on one side thereof, and the collection receptacle is open through an aspiration/drain port 26. The wall 20 further defines an airflow passage 30 which is open at its lower end and which proceeds along the path shown by arrow 32 past the wall and baffle 22. In this way, airflow through the collection device 10 an continue in a relatively unimpeded manner while secretions will be diverted into the receptacle 24 by the diverter structure 22.

The collection device 10 will be “collapsible” into a relatively low diameter configuration and expandable from the low diameter configuration to a deployed configuration which is typically sized slightly larger than the trachea of the patient being treated. The body 12 may be elastic so that it can be collapsed by external constraint and allowed to self-expand into its deployed configuration. Alternatively, the body 12 may be expandable by inflation of an internal balloon or use of another expandable delivery device.

Referring now to FIGS. 3A-3D, the sub-glottic secretion collection device 10 may be deployed in a patient's trachea T by first forming an incision I in the patient's neck into the trachea below the larynx L. A guidewire GW is then introduced through the incision I upwardly past the larynx and outwardly through the patient's mouth.

As shown in FIG. 3B, a delivery catheter 40 may then be used to introduce the collection device 10 over the guidewire GW, through the patient's mouth and into the trachea T below the larynx L. The guidewire will be passed through the port 26 of the device 10 so that the port will enter the incision I before the remainder of the device is deployed.

Referring now to FIG. 3C, after the port 26 has been drawn through the incision I, the delivery catheter 40 may be withdrawn, allowing the collection device 10 to be deployed within the trachea T. As shown in FIG. 3C, the device is self-expanding. It will be appreciated that a separate balloon or other expansion structure could be provided in order to deploy the device in alternative protocols.

Referring now to FIG. 3D, once the device 10 has been deployed in the trachea T, an aspiration source 50 may be connected to the aspiration port 26 by tubing 52. Thus, secretions S which collect in the receptacle 24 may be continuously withdrawn by aspiration while the patient remains able to breathe through the mouth via the unobstructed airflow passage 32.

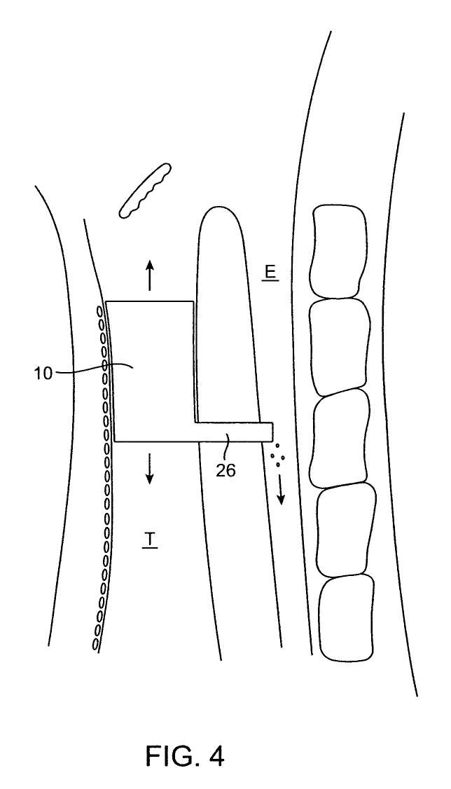

While the preferred method of the present invention will rely on continuous aspiration through port 26, as illustrated in FIG. 3D, in some instances it may be possible to provide for passive collection and drainage of secretions into the patient's esophagus E, as shown in FIG. 4. In that instance, the port 26 will be placed through a fistula formed between the trachea T and esophagus, as illustrated. The patient will still be able to breathe through the defined airway while the collected secretions will flow directly into the esophagus and ultimately into the patient's stomach.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A sub-glottic secretion collection device comprising:
a body expandable within the trachea beneath the larynx, said body defining an air flow path and a secretions receptacle;
a baffle within the body to direct secretions toward the receptacle and away from the air flow path; and
a port open to the receptacle to allow secretions to drain or be aspirated from the receptacle.

2. A sub-glottic device as in claim 1, wherein the body s a self-expanding scaffold that can be delivered in a narrow width configuration and allowed to expand in situ within the trachea.

3. A sub-glottic device as in claim 1, wherein the scaffold is malleable so that it may be expanded by a radially outward force.

4. A method for delivering the sub-glottal device of claim 1 to a patient, said method comprising:
positioning a guidewire through the patient's mouth, into the trachea and out through an external incision or through an incision from the trachea to the esophagus;
passing the device over the guidewire and into the trachea in a narrow width configuration; and
expanding the device in situ so that the body anchors in the trachea with the port passing through the incision.

5. A method for collecting secretions passing down a patient's trachea, said method comprising:
implanting a diverter device in the trachea below the larynx;
allowing secretions to collect in a receptacle positioned in the diverter device; and
allowing air inspiration and exhalation through a flowpath past the diverter.

6. A method as in claim 5, wherein the collected secretions are allowed to flow externally through a port.

7. A method as in claim 5, wherein the collected secretions are allowed to flow into the esophagus through a port.

8. A method as in claim 5, wherein the secretions are periodically aspirated through a port.
